(19) United States
(12) Patent Application Publication
(10) Pub. No.: US 2004/0127797 A1
(43) Pub. Date: Jul. 1, 2004

(54) System and method for measuring bladder wall thickness and presenting a bladder virtual image

(76) Inventors: Bill Barnard, Woodinville, WA (US); Stephen Dudycha, Bothell, WA (US); Vikram Chalana, Mill Creek, WA (US); Gerald McMorrow, Kirkland, WA (US); Jongtae Yuk, Redmond, WA (US)

Correspondence Address: BLACK LOWE & GRAHAM, PLLC, 701 FIFTH AVENUE, SUITE 4800, SEATTLE, WA, 98104, US

(21) Appl. No.: 10/704,996

(22) Filed: Nov. 10, 2003

Related U.S. Application Data
(63) Continuation-in-part of application No. 10/165,556, filed on Jun. 7, 2002, now Pat. No. 6,676,605.
(63) Continuation-in-part of application No. PCT/US03/24368, filed on Aug. 1, 2003.
(63) Continuation-in-part of application No. PCT/US03/14785, filed on May 9, 2003.
Application No. PCT/US03/14785 is a continuation of application No. 10/165,556, filed on Jun. 7, 2002, now Pat. No. 6,676,605.
(63) Continuation-in-part of application No. 10/633,186, filed on Jul. 31, 2003.
(60) Provisional application No. 60/423,881, filed on Nov. 5, 2002.
(60) Provisional application No. 60/400,624, filed on Aug. 2, 2002.
(60) Provisional application No. 60/470,525, filed on May 12, 2003.
(60) Provisional application No. 60/400,624, filed on Aug. 2, 2002.
(60) Provisional application No. 60/423,881, filed on Nov. 5, 2002.

(51) Int. Cl. A61B 8/02

(52) U.S. Cl. 600/449

(57) ABSTRACT

An ultrasound transceiver scans a bladder in a three dimensional array to measure the thickness and surface area of the bladder to determine bladder mass. The bladder wall thickness and masses may be determined for anterior, posterior, and lateral locations of the bladder.

### PRIORITY CLAIM

This application is a continuation-in-part of and claims priority to U.S. patent application filed Nov. 5, 2003 via U.S. Postal Service Express Mail number EL962633890US, which claims priority to U.S. patent application Ser. No. 10/633,186 filed Jul. 31, 2003 which claims priority to U.S. patent application Ser. No. 10/443,126 filed May 12, 2003, which claims priority to U.S. provisional patent application serial No. 60/423,881 filed Nov. 5, 2002 and U.S. provisional patent application serial No. 60/400,624 filed Aug. 2, 2002.

This application is also a continuation-in-part of and claims priority to U.S. Patent application Ser. No. 10/165,556 filed Jun. 7, 2002.

This application is also a continuation-in-part of and claims priority to PCT application serial number PCT/US03/24368 filed Aug. 1, 2003, which claims priority to U.S. provisional patent application serial No. 60/423,881 filed Nov. 5, 2002 and U.S. provisional patent application serial No. 60/400,624 filed Aug. 2, 2002.

This application is also a continuation-in-part of and claims priority to PCT Application Serial No. PCT/US03/14785 filed May 9, 2003, which is a continuation of U.S. patent application Ser. No. 10/165,556 filed Jun. 7, 2002.

This application is also a continuation-in-part of and claims priority to U.S. patent application Ser. No. 10/633,186 which claims priority to U.S. provisional patent application serial No. 60/423,881 filed Nov. 5, 2002 and U.S. provisional patent application serial No. 60/423,881 filed Aug. 2, 2002, and to U.S. patent application Ser. No. 10/443,126 filed May 20, 2003 which claims priority to U.S. provisional patent application serial No. 60/423,881 filed Nov. 5, 2002 and to U.S. provisional application 60/400,624 filed Aug. 2, 2002.

This application also claims priority to U.S. provisional patent application serial No. 60/470,525 filed May 12, 2003, and to U.S. patent application Ser. No. 10/165,556 filed Jun. 7, 2002. All of the above applications are herein incorporated by reference in their entirety as if fully set forth herein.

### FIELD OF THE INVENTION

This invention relates generally to using ultrasound in diagnosing bladder condition or dysfunction.

### BACKGROUND OF THE INVENTION

A variety of techniques have been used to evaluate bladder dysfunction. Such techniques typically attempt to determine the size of the bladder or bladder volume, meaning the amount of urine in the bladder. As one example, U.S. Pat. No. 6,110,111 to Barnard discloses a system for assessing bladder distension by using ultrasound to compare the bladder surface area with the surface area of a sphere. According to Barnard, the closer the bladder is to a spherical shape, the greater the pressure within the bladder.

Bladder mass measurements can also be used to diagnose several different clinical conditions. Bladder wall thickness and bladder mass can be used to indicate bladder outlet obstruction and bladder distension. An outlet obstruction will cause a higher pressure in the urine, against which the bladder muscle must contract. That higher pressure causes the muscle to exert more force, resulting in hypertrophy of the bladder muscle. Symptoms of bladder muscle hypertrophy include increased wall thickness and increased mass. The use of bladder wall thickness as an indicator of detrusor hypertrophy has been noted for many years (see Matthews P N, Quayle J B, Joseph A E A, Williams J E, Wilkinson K W, Riddle P R, The use of ultrasound in the investigation of prostatism, British Journal of Urology, 54:536-538, 1982; and Cascione C J, Bartone F F, Hussain M B, Transabdominal ultrasound versus excretory urography in preoperative evaluation of patients with prostatism, Journal of Urology, 137:883-885, 1987). Converting bladder wall thickness to bladder wall volume (or bladder mass by multiplying bladder wall volume by the specific gravity of bladder tissue) yields a single number, which is independent of bladder volume. While the bladder wall thins as volume increases, the total bladder wall volume (or bladder mass) remains unchanged.

Another key parameter of bladder functionality is bladder distension. As the bladder volume and bladder pressure increases, the bladder walls stretch and thin. Two prominent maladies associated with bladder distension are incontinence and hyperdistension.

Incontinent episodes frequently occur if the bladder sphincter muscles are unable to retain urine as bladder pressure and bladder distension increases. In many individuals, this incontinent point occurs at a consistent volume. Consequently, if this volume is known and if the bladder volume can be measured over time, then incontinent events can be prevented. Furthermore, research has shown that it is possible to increase both the bladder capacity and the bladder volume incontinent point through a variety of methods. This technique has been used effectively on enuretic patients.

Hyperdistension refers to the case in which the bladder is allowed to fill to such an extreme that excessive bladder pressure builds which can cause potential renal damage, renal failure and even patient death from autonomic dysreflexia if the patient has spinal cord damage. As with incontinence, hyperdistension has been successfully prevented using non-invasive bladder volume measuring.

At small bladder volumes, bladder response is quite constant across humanity. Normal adult humans typically have no trouble voiding and leaving less than 50 ml of urine. Thus, it has been relatively easy to establish post-void-residual (PVR) volumes that are normal and PVR volumes that are potential medical problems. At low bladder volumes, bladder distension information is not as useful. However, normal humans have widely variant bladder capacities. Thus, it is more difficult to establish a volume threshold at which over-distension occurs or when incontinence occurs. As the bladder fills, quantization of bladder distension becomes more useful. This is especially true since it is thought that a bladder distension metric would better indicate hyperdistension and bladder capacity.

Current methods to measure bladder wall thickness rely on one-dimensional (A-mode) and two-dimensional (B-mode) ultrasound and are greatly susceptible to operator error, time consuming, and inaccurate. The operator using one or two-dimensional ultrasound has to repeatedly reposition the ultrasound probe until a bladder wall image is sufficiently visible, usually the more anterior portion of the bladder. Furthermore, the limitations of one and two-dimensional ultrasound require inaccurate spherical model assumptions for the bladder. Presumably for these and other reasons, the industry has concluded that measuring bladder wall thickness is an unreliable or ineffective means to quantize bladder distension. See, e.g., Barnard, U.S. Pat. No. 6,110,111 at column 1, lines 50-59.

Thus, there is a need for a system to accurately measure bladder wall thickness for use in evaluating bladder distension.

### SUMMARY OF THE INVENTION

The present invention incorporates a three-dimensional ultrasound device to scan a patient's bladder. Data collected in the ultrasound scan are presented in an array of 2D scanplanes and in a substantially bas-relief 2D presentation of bladder hemispheres showing the bladder wall. The collected data is analyzed to calculate bladder thickness and mass. Bladder mass information is then used to assess bladder dysfunction.

In accordance with the preferred embodiment of the invention, a microprocessor-based ultrasound apparatus, placed on the exterior of a patient, scans the bladder of the patient in multiple planes with ultrasound pulses, receives reflected echoes along each plane, transforms the echoes to analog signals, converts the analog signals to digital signals, and downloads the digital signals to a computer system.

Although a variety of scanning and analysis methods may be suitable in accordance with this invention, in a preferred embodiment the computer system performs scan conversion on the downloaded digital signals to obtain a three-dimensional, conically shaped image of a portion of the bladder from mathematical analysis of echoes reflecting from the inner (submucosal) and outer (subserosal) surfaces of the bladder wall. The conical image is obtained via three-dimensional C-mode ultrasound pulse echoing using radio frequency (RF) ultrasound (approximately 2-10 MHz) to obtain a 3D array of 2D scanplanes, such that the scanplanes may be a regularly spaced array, an irregular spaced array, or a combination of a regularly spaced array and irregularly spaced array of 2D scanplanes. The 2D scanplanes, in turn, are formed by an array of one-dimensional scanlines (ultrasound A-lines), such that the scanlines may be regularly spaced, irregularly spaced, or a combination of regularly spaced and irregularly spaced scanlines. The 3D array of 2D scanplanes results in a solid angle scan cone.

Alternatively, a solid angle scan cone is obtained by 3D data sets acquired from a three-dimensional ultrasound device configured to scan a bladder in a 3D scan cone of 3D distributed scanlines. The 3D scan cone is not a 3D array of 2D scanplanes, but instead is a solid angle scan cone formed by a plurality of internal and peripheral one-dimensional scanlines. The scanlines are ultrasound A-lines that are not necessarily confined within a scanplane, but would otherwise occupy the inter-scanplane spaces that are in the 3D array of 2D scanplanes.

The solid angle scan cones, either as a 3D array of 2D scanplanes, or as a 3D scan cone of 3D distributed scanlines, provides the basis to locate bladder wall regions or surface patches of the inner and outer surfaces of the bladder wall. The location of each surface patch is determined using fractal analytical methods and the distance or thickness between the inner and outer surface patches is measured. The bladder wall mass is calculated as a product of the surface area of the bladder, the bladder wall thickness, and the specific gravity of the bladder wall. The entire bladder wall or various regions, including anterior, posterior, and lateral portions of the bladder, may be measured for thickness and mass.

An alternate embodiment of the invention configures the downloaded digital signals to be compatible with a remote microprocessor apparatus controlled by an Internet web-based system. The Internet web-based system has multiple programs that collect, analyze, and store organ thickness and organ mass determinations. The alternate embodiment thus provides an ability to measure the rate at which internal organs undergo hypertrophy over time. Furthermore, the programs include instructions to permit disease tracking, disease progression, and provide educational instructions to patients.

Another embodiment of the invention presents the bladder, obtained from the 3D array of 2D scanplanes or the 3D scan cone of 3D distributed scanlines, in a substantially 2D bas relief image. The effect is to have the three-dimensional ultrasound device function as a virtual cystoscope. The bas-relief image presents the bladder in cross sectional hemispheres, where the bladder, the bladder wall thickness, and structures in the bladder and bladder wall are visible as a virtual 3D-like image. The virtual bas-relief image is obtained by remote, non-intrusive ultrasound scans processed to present a similar image that would otherwise be obtained by an intrusive, visible light cystoscope.

### BRIEF DESCRIPTION OF THE DRAWINGS

The preferred and alternative embodiments of the present invention are described in detail below with reference to the following drawings.

FIG. 1 is a microprocessor-controlled transceiver;

FIG. 2 is a representation of scanlines sharing a common rotational angle to form a plane;

FIG. 3 is a side view representation of a collection of scanplanes that are separated by approximately 7.5 degrees from each other;

FIG. 4 is a top view representation of a collection of planes, each rotated 7.5 degrees from each other;

FIG. 5 is a graphical representation of a plurality of 3D distributed scanlines emanating from the transceiver forming a scan cone;

FIG. 6 is an algorithm for measuring bladder thickness and mass;

FIG. 7 is a representation of four surface patch elements, each constructed from the sixteen neighboring points that surround the patch;

FIG. 8 is a representation of three scanlines passing through the subserosal and submucosal wall locations of the bladder; and

FIG. 9 depicts a substantially bas-relief 2D presentation volume rendering of the left and right half bladder hemisphere views of a bladder.

### DETAILED DESCRIPTION OF THE PREFERRED EMBODIMENT

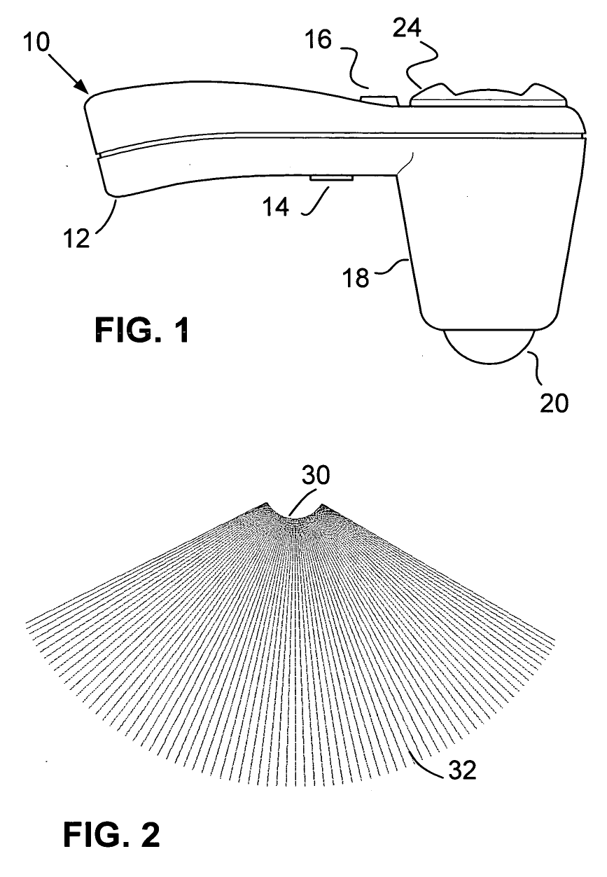

The portable embodiment of the ultrasound transceiver of the present invention is shown in FIG. 1. The transceiver 10 includes a handle 12 having a trigger 14 and a gender changer 16, a transceiver housing 18 attached to the handle 12, a transceiver dome 20 and a display 24 for user interaction, attached to the transceiver housing 18 at an end opposite the transceiver dome 20. The transceiver 10 is held in position against the body of a patient by a user. In operation, the transceiver transmits a radio frequency ultrasound signal within the 2 to 10 MHz range to the body and then receives a returning echo signal. The returning echo signal provides an image signal for image processing. The gender changer 16 serves to adjust the delivery and reception of radio frequency ultrasound to the anatomy of a male patient and to the anatomy of a female patient. The transceiver is controlled by a microprocessor and software associated with the microprocessor and a digital signal processor of a computer system. As used in this invention, the term “computer system” broadly comprises any microprocessor-based or other computer system capable of executing operating instructions and manipulating data, and is not limited to a traditional desktop or notebook computer. The display 24 presents alphanumeric data indicating the proper or optimal positioning of the transceiver 10 for initiating a series of scans. In alternate embodiments, the two- or three-dimensional image of a scanplane may be presented in the display 24 of FIG. 1.

Although the preferred ultrasound transceiver is described above and depicted in FIG. 1, other transceivers may also be used. For example, the transceiver need not be battery-operated or otherwise portable, need not have a top-mounted display 24, and may include many other features or differences. The transceiver 10 need only be able to non-invasively probe within the body to gather data that can be used to analyze internal objects such as the bladder. The display 24 may be a liquid crystal display (LCD), a light emitting diode (LED), a cathode ray tube (CRT), or any suitable display capable of presenting alphanumeric data or graphic images.

Once optimally positioned over the abdomen for scanning, the transceiver 10 transmits an ultrasound signal (approximately 3.7 MHz in the preferred embodiment, and commonly in a 2-10 MHZ range) into the bladder region. The ultrasound signal is in the form of generally linear signal bursts known as scanlines, as illustrated in FIG. 2. The scanlines, each approximately 20 cm long, originate from the transceiver dome 20, producing a dome cutout 30 in a cluster of scanlines forming a scanplane 32. Within the scanplane 32 are a plurality of scanlines that share a common rotational angle (θ), but have a unique tilt angle (φ). In the preferred embodiment, each plane contains 77 scan lines, although the number of lines can vary within the scope of this invention, and the angular separation between the lines can vary within the scope of this invention.

The angular separation or spacing between lines may be uniform (substantially equal angular spacings, say 1.5° between each scanline) or non-uniform (substantially unequal angular spacings). An example of non-uniform angular spacing would be “1.5-6.8-15.5-7.2-so on” sequence where 1.5° is between a first line and a second line, 6.8° is between the second line and a third line, 15.5° is between the third line and a fourth line, 7.2° is between the fourth line and a fifth line, and so on. The angular separation may also be a combination of uniform and non-uniform angular spacings, for example a sequence of “1.5-1.5-1.5-7.2-14.3-20.2-8.0-8.0-8.0-4.3-7.8-so on” angular spacings.

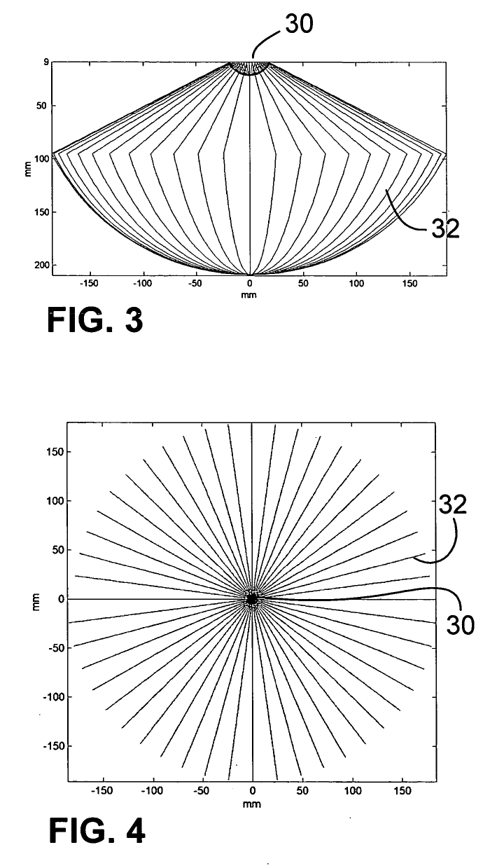

After a plane of scanlines is transmitted, the transceiver rotational angle θ is incremented slightly and another plane of pulse-echo signals are transmitted and received to form a new scanplane. This process is repeated as desired, producing a series of scanplanes in which each plane will be slightly rotated from the prior plane by a selected rotational angle θ interval. The rotational angle θ interval or spacing between scanplanes can be uniform or nonuniform. Uniform intervals between scanplanes have approximately the same degrees separating each scanplane from its nearest neighbors. For example, as shown in FIG. 3, in the preferred embodiment each scanplane 32 is transmitted, received, and displayed into a twenty-four plane array, with approximately 7.5° rotational angle θ interval separating each scanplane from its nearest neighbors in the array. In contrast, an example of non-uniform intervals between scanplanes in an array having a sequence “3.0-18.5-10.2-so on” would be a rotational angle θ interval of 3.0° between a first and a second scanplane, a θ interval of 18.5° between the second scanplane and a third scanplane, then a θ interval of 10.2° between the third scanplane and a fourth scanplane, and so on. The scanplane interval may also be a combination of uniform and non-uniform rotational angle θ intervals, for example a sequence of “3.0-3.0-3.0-18.5-10.2-20.6-7.5-7.5-7.5-16.0-5.8-so on” θ intervals. Also illustrated in FIG. 3 is the tilt angle φ that sweeps through angles between −60° and 60° for a total of 120°.

FIG. 4 presents a top view of a twenty-four plane array, the twenty-four array having a uniform rotational angle θ between each scanplane. The number of scanplanes in the array is at least two, but can be varied above two. The rotational angle θ intervals between scanplanes in an array can be varied, and be uniform and non-uniform.

For wedge and translational arrays, the scanplanes may similarly be uniformly spaced, non-uniformly spaced, or a combination of uniformly spaced and non-uniformly spaced scanplanes.

As the scanlines are transmitted and received, the returning echoes are changed into analog electrical signals by a transducer, converted to digital signals by an analog-to-digital converter, and conveyed to the digital signal processor of the computer system for analysis to determine the locations of the bladder walls. The computer system itself is not depicted, but in a preferred embodiment includes a microprocessor and a RAM, hard-drive, optical drive, or other memory for storing processing instructions and data generated by the transceiver 10.

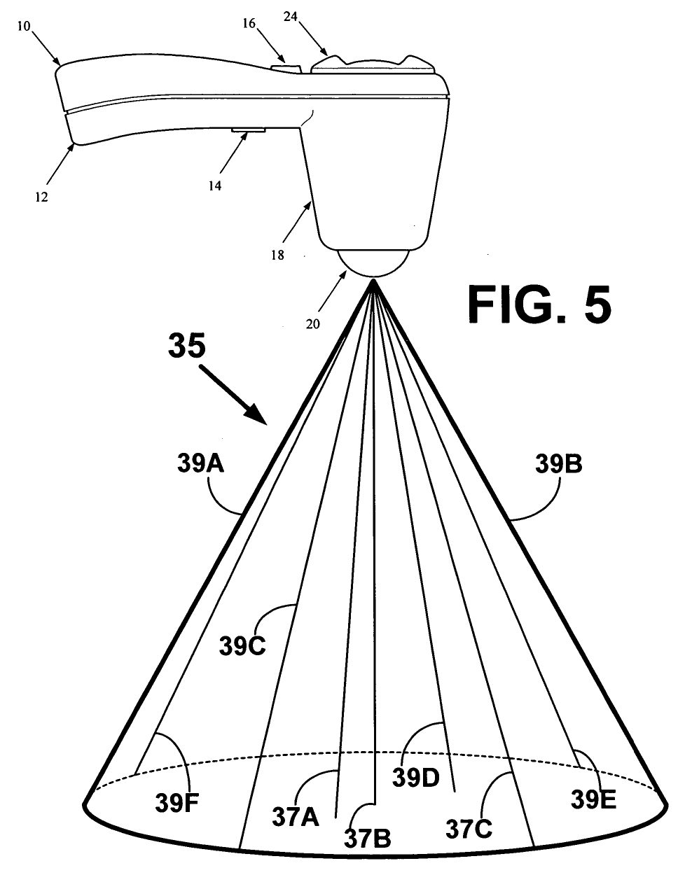

FIG. 5 is a graphical representation of a plurality of 3D-distributed scanlines emanating from the transceiver 10 forming a scan cone 35. The scan cone 35 is formed by a plurality of 3D distributed scanlines that comprises a plurality of internal and peripheral scanlines. The scanlines are one-dimensional ultrasound A-lines that emanate from the tranciever 10 at different coordinate directions, that taken as an aggregate, from a conic shape. The 3D-distributed A-lines (scanlines) are not necessarily confined within a scanplane, but instead are directed to sweep throughout the internal and along the periphery of the scan cone 35. The 3D-distributed scanlines not only would occupy a given scanplane in a 3D array of 2D scanplanes, but also the inter-scanplane spaces, from the conic axis to and including the conic periphery. The transceiver 10 shows the same illustrated features from FIG. 1, but is configured to distribute the ultrasound A-lines throughout 3D space in different coordinate directions to form the scan cone 35.

The internal scanlines are represented by scanlines 37A-C. The number and location of the internal scanlines emanating from the transceiver 10 is the number of internal scanlines needed to be distributed within the scan cone 35, at different positional coordinates, to sufficiently visualize structures or images within the scan cone 35. The internal scanlines are not peripheral scanlines. The peripheral scanlines are represented by scanlines 39A-F and occupy the conic periphery, thus representing the peripheral limits of the scan cone 35.

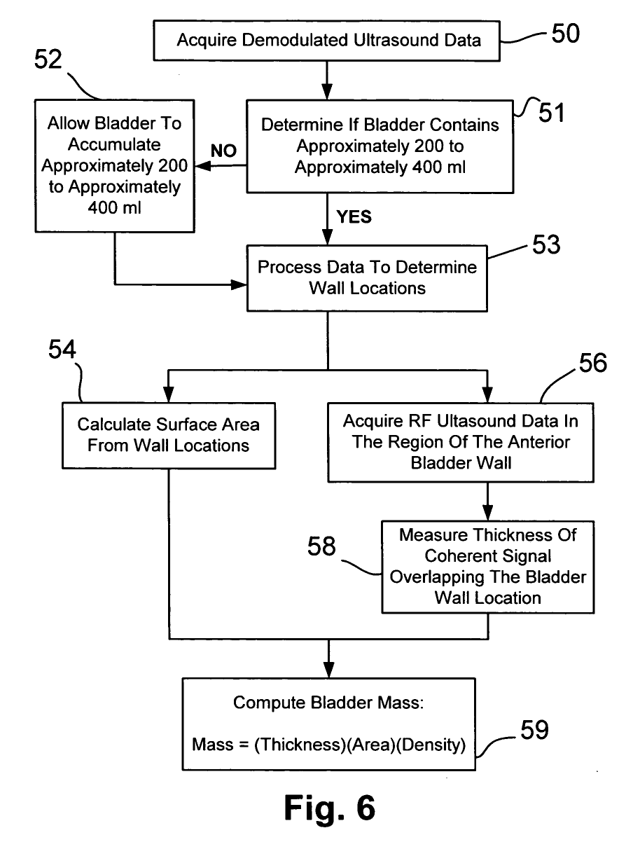

Once the wall locations are identified, the wall locations, demodulated magnitude data, and a subset of quadrature amplitude demodulated signal in the region of the anterior bladder wall are directed to the microprocessor for further analysis according to the algorithm illustrated in FIG. 6 for the preferred embodiment of the invention. First, ultrasound data is acquired relative to the bladder, as shown in the first block 50. In general, bladder-specific data can be acquired by a user who manipulates the transceiver 10 while viewing the received data on a display screen and then positioning the transceiver 10 as necessary so that the bladder is sufficiently within the field of view of the cone as depicted in FIG. 3, or within the field of view of the scan cone 35 depicted in FIG. 5.

After obtaining ultrasound bladder data, the ultrasound data is processed to determine if the bladder contains approximately 200 to approximately 400 ml, as shown in the second block 51. If “No”, then the bladder is allowed to accumulate approximately 200 to approximately 400 ml, as shown in the third block 52, or, if “Yes, meaning the bladder already contains the preferred approximate 200-400 ml volume, then the locations of the bladder walls, as shown in the fourth block 53, may be undertaken. The determination of organ wall locations and other such exterior boundaries within an ultrasound scan is within the capability of ultrasound devices presently on the market. In general, however, the process determines the length of a scanline from the transceiver dome to the bladder wall. The data, including wall locations, is stored in the computer memory.

Once the full cone of ultrasound magnitude data has been scanned and wall locations have been determined by the digital signal processor, the microprocessor further analyzes the data to correct any misdetection in wall location and to determine bladder volume. Two specific techniques for doing so are disclosed in detail in U.S. Pat. No. 4,926,871 to Ganguly et al and U.S. Pat. No. 5,235,985 to McMorrow et al, which are incorporated by reference. These patents provide detailed explanations for non-invasively transmitting, receiving and processing ultrasound signals relative to the bladder, and then for calculating bladder volume.

Using the methods provided by the '871 and '985 patents, the resultant data is used to determine whether or not the bladder volume is with a range of approximately 200 to approximately 400 ml. If the bladder volume is within that range, the ultrasound data is used to determine the actual surface area from the wall locations, as indicated in the fifth block 54. The surface area calculation is explained in greater detail below. While calculating the surface area in the fifth block 54, reflected RF ultrasound waves are received from the anterior bladder wall, as indicated in the sixth block 56. Although these tasks are preferably conducted in parallel, they may alternatively be processed in series. Thereafter, as shown in the seventh block 58, the bladder wall thickness is determined from the coherent signals that overlap at the wall locations. The determination of bladder wall thickness is explained in greater detail below. Finally, as shown in the seventh block 58, the bladder mass is computed as a product of thickness, area, and bladder density.

The volume restriction described in the previous paragraph defines the range of bladder volumes that enable an optimal measurement of the bladder mass. The mass calculation may be performed at a volume not in this range, but this will generally result in a less accurate measurement. For example, bladder volumes less than 200 ml and greater than 400 ml can be measured, but with less accuracy. For volumes substantially greater than 400 ml, for example bladder volumes of 1000 ml to multi-liters, the preferred embodiment will utilize scanlines greater than 20 cm to accommodate the larger bladder sizes. The preferred embodiment may be applied to measure the thicknesses and masses of internal organs of human and animals. The lengths of the scanlines are adjusted to match the dimensions of the internal organ to be scanned.

Surface area determination. The surface area measurement of fifth block 54 is performed by integrating the area of interpolating surface patch functions defined by the wall locations. The mathematical calculations are provided below in greater detail.

The surface of the bladder is defined to be S. This surface corresponds to the actual surface of the bladder determined by analysis of the wall locations of the bladder. Since this shape is not known in advance, modeling the bladder as a sphere or an ellipsoid provides only a crude approximation of the surface. Instead, the surface S is defined as a construction of a series of individual surface patches sᵢⱼ, where i and j count through the latitude and longitude components of the surface, similar to the division of the Earth's surface into lines of latitude and longitude. The area of the bladder surface, S, is defined as the sum of all the individual surface patches, S=Σsᵢⱼ.

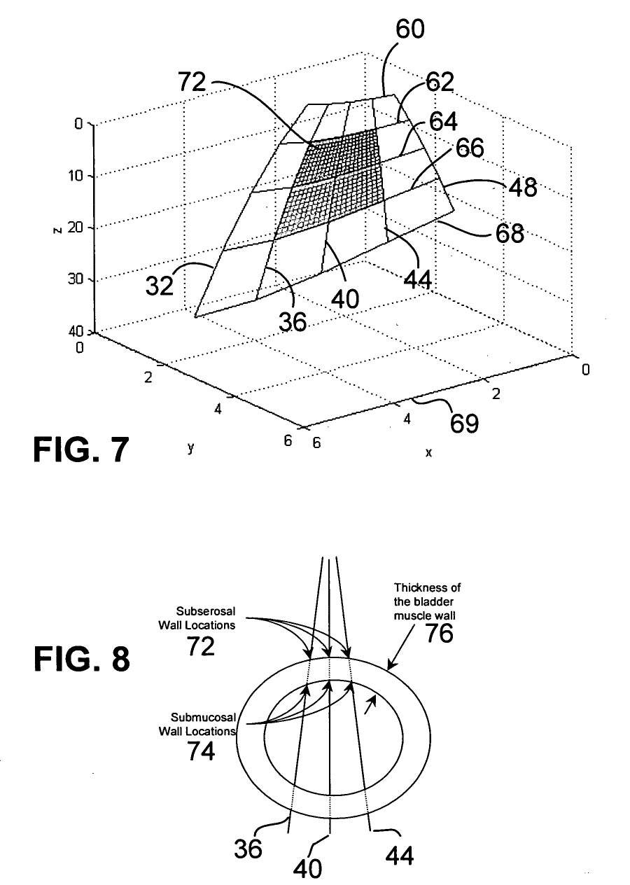

As depicted in three dimensions in FIG. 7, by way of example, five scanplanes 32-48 are seen transmitted substantially longitudinally across a subserosal wall location 72 referenced to a tri-axis plotting grid 69. The five scanplanes include the first scanplane 32, a second scanplane 36, a third scanplane 40, a fourth scanplane 44, and a fifth scanplane 48. The scanplanes are represented in the preceding formulas as subscripted variable j. Substantially normal to the five longitudinal scanplanes are five latitudinal integration lines 60-68 that include a first integration line 60, a second integration line 62, a third integration line 64, a fourth integration line 66, and a fifth integration line 68. The integration lines are represented in the preceding formulas as subscripted variable i.

By way of example, four surface patch functions are highlighted in FIG. 7 as the subserosal wall location 72. The i and j subscripts mentioned previously correspond to indices for the lines of latitude and longitude of the bladder surface. For the purposes of this discussion, i will correspond to lines of longitude and j will correspond to lines of latitude although it should be noted the meanings of i and j can be interchanged with a mathematically equivalent result. Using the scanplane and integration line definitions provided in FIG. 7, the four surface patch functions are identified, in the clockwise direction starting in the upper left, as s_{36,62}, s_{40,62}, s_{40,64}, and s_{36,64}.

The surface patches are defined as functions of the patch coordinates, sᵢⱼ(u,v). The patch coordinates u and v, are defined such that 0≦u, v<1 where 0 represents the starting latitude or longitude coordinate (the i and j locations), and 1 represents the next latitude or longitude coordinate (the i+1 and j+1 locations). The surface function could also be expressed in Cartesian coordinates where sᵢⱼ(u,v)=xᵢⱼ(u,v)i+yᵢⱼ(u,v)j+zᵢⱼ(u,v)k where i, j, k, are unit vectors in the x-, y-, and z-directions respectively. In vector form, the definition of a surface patch function is given in Equation 1.

$${s}_{i , j} ( u , v ) = [ \begin{matrix}{x}_{i , j} ( u , v ) \\ {y}_{i , j} ( u , v ) \\ {z}_{i , j} ( u , v )\end{matrix} ] . \qquad \text{Equation 1}$$

With the definitions of surface patch functions complete, attention can turn to the surface area calculation represented in the fifth block 54 of FIG. 6. The surface area of S, A(S), can be defined as the integration of an area element over the surface S, as shown in Equation 2. Since S is composed of a number of the patch surface functions, the calculation for the area of the surface S can be rewritten as the sum of the areas of the individual surface patch functions as in Equation 3.

$$\begin{matrix}A ( S ) = {\int }_{s} ⅆ A . & \text{Equation 2} \\ A ( S ) = {\sum }_{i , j} A ( {s}_{i , j} ) . & \text{Equation 3}\end{matrix}$$

Similarly, to Equation 2 for the entire surface, the area of the surface patch is the integration of an area element over the surface patch, shown in Equation 4. The integration over the surface patch function can be simplified computationally by transforming the integration over the surface to a double integration over the patch coordinates u and v. The transformation between the surface integration and the patch coordinate integration is shown in Equation 5.

$$\begin{matrix}A ( {s}_{i , j} ) = {\int }_{{s}_{i , j}} ⅆ {A}_{i , j} . & \text{Equation 4} \\ {\int }_{{s}_{i , j}} ⅆ {A}_{i , j} = {\int }_{u = 0}^{1} {\int }_{v = 0}^{1} \vert \frac{\partial {s}_{i , j}}{\partial u} \times \frac{\partial {s}_{i , j}}{\partial v} \vert ⅆ v ⅆ u . & \text{Equation 5}\end{matrix}$$

By substituting Equation 5 into Equation 4, and Equation 4 into Equation 3, the area for the entire surface can be calculated. The result of these substitutions is shown in Equation 6.

$$A ( S ) = {\sum }_{i , j} {\int }_{u} {\int }_{v} \vert \frac{\partial {s}_{i , j}}{\partial u} \times \frac{\partial {s}_{i , j}}{\partial v} \vert ⅆ v ⅆ u . \qquad \text{Equation 6}$$

The surface patch function may be any function that is continuous in its first derivatives. In the embodiment shown, a cubic B-spline interpolating function is used for the interpolating surface patch function although any surface function may be used. This interpolating function is applied to each of the Cartesian coordinate functions shown in Equation 1. The interpolating equation for the x-coordinate of the sᵢⱼ patch function is given in Equation 7. Similar calculations are performed for the yᵢⱼ and zᵢⱼ components of the surface patch function.

Equation 7.

xᵢⱼ(u,v)=uM_{b}X_{i,j}M_{b}^{t}v^{t}

where t denotes matrix and vector transpose,

$$u = [ \begin{matrix}{u}^{3} \\ {u}^{2} \\ u \\ 1\end{matrix} ] , v = [ \begin{matrix}{v}^{2} \\ {v}^{2} \\ v \\ 1\end{matrix} ] , {M}_{b} [ \begin{matrix}- 1 & 3 & - 3 & 1 \\ 3 & - 6 & 3 & 0 \\ - 3 & 0 & 3 & 0 \\ 1 & 4 & 1 & 0\end{matrix} ] , and$$

$${X}_{i , j} = [ \begin{matrix}{x}_{i - 1 , j - 1} & {x}_{i - 1 , j} & {x}_{i - 1 , j + 1} & {x}_{i - 1 , j + 2} \\ {x}_{i , j - 1} & {x}_{i , j} & {x}_{i , j + 1} & {x}_{i , j + 2} \\ {x}_{i + 1 , j - 1} & {x}_{i + 1 , j} & {x}_{i + 1 , j + 1} & {x}_{i + 1 , j + 2} \\ {x}_{i + 2 , j - 1} & {x}_{i + 2 , j} & {x}_{i + 2 , j + 1} & {x}_{i + 2 , j + 2}\end{matrix} ]$$

Since the interpolating functions for each of the patch functions is a cubic surface, the integration may be performed exactly using a quadrature formula. The formula used in this application is shown in Equation 8.

$$A ( {s}_{i , j} ) = {\sum }_{i , j} \frac{1}{4} ( \begin{matrix}{\vert \frac{\partial {s}_{i , j}}{\partial u} \times \frac{\partial {s}_{i , j}}{\partial v} \vert}_{u = \frac{3 - \sqrt{3}}{6} , v = \frac{3 - \sqrt{3}}{6}} + \\ {\vert \frac{\partial {s}_{i , j}}{\partial u} \times \frac{\partial {s}_{i , j}}{\partial v} \vert}_{u = \frac{3 - \sqrt{3}}{6} , v = \frac{3 + \sqrt{3}}{6}} + \\ \begin{matrix}{\vert \frac{\partial {s}_{i , j}}{\partial u} \times \frac{\partial {s}_{i , j}}{\partial v} \vert}_{u = \frac{3 + \sqrt{3}}{6} , v = \frac{3 - \sqrt{3}}{6}} + \\ {\vert \frac{\partial {s}_{i , j}}{\partial u} \times \frac{\partial {s}_{i , j}}{\partial v} \vert}_{u = \frac{3 + \sqrt{3}}{6} , v = \frac{3 + \sqrt{3}}{6}}\end{matrix}\end{matrix} ) . \qquad \text{Equation 8}$$

Recalling the fact that sᵢⱼ(u,v) is defined as a vector function in Cartesian coordinates (Equation 1), the norm of the cross product of the partial derivatives can be written as follows:

$$\vert \frac{\partial {s}_{i , j}}{\partial u} \times \frac{\partial {s}_{i , j}}{\partial u} \vert = \sqrt{\begin{matrix}{( \frac{\partial {y}_{i , j}}{\partial u} \frac{\partial {z}_{i , j}}{\partial v} - \frac{\partial {z}_{i , j}}{\partial u} \frac{\partial {y}_{i , j}}{\partial v} )}^{2} + \\ {( \frac{\partial {z}_{i , j}}{\partial u} \frac{\partial {x}_{i , j}}{\partial v} - \frac{\partial {z}_{i , j}}{\partial u} \frac{\partial {x}_{i , j}}{\partial v} )}^{2} + \\ {( \frac{\partial {x}_{i , j}}{\partial u} \frac{\partial {y}_{i , j}}{\partial v} - \frac{\partial {y}_{i , j}}{\partial u} \frac{\partial {x}_{i , j}}{\partial v} )}^{2}\end{matrix}} . \qquad \text{Equation 9}$$

When the physical x-, y-, and z-locations are used in the interpolating function, the surface are will be calculated in the square of the units of x, y, and z. At this point, the calculation in the fifth block 54 of FIG. 6 is complete.

Wall thickness determination. The second component to the mass calculation is a measurement of the thickness of the bladder muscle wall. This thickness is defined to be the normal thickness between the subserosal and submucosal surfaces of the bladder wall.

The wall thickness is calculated from the fractal dimension of the RF signal in the region of the wall thickness. The fractal dimension increases due to the multiplicity of interface reflections through the bladder muscle. The increase and decrease of fractal dimension through the bladder muscle wall can be modeled as a parabola where the fractal dimension is a function of the depth in the region of the bladder wall. The thickness of the bladder is then determined to be the region of the parabola model that is at least 97% of the maximal value of the fractal dimension. The calculations are reviewed below in Equation 10.

$${fd}_{r} = \frac{log ( \frac{\begin{matrix}max ( {RF}_{r = r - w / 2 , r + w / 2} ) - \\ min ( {RF}_{r = r - w / 2 , r + w / 2} ) + w\end{matrix}}{w} )}{log ( \frac{n}{w} )} . \qquad \text{Equation 10}$$

The fractal dimension calculation corresponds to the fourth block 56 of FIG. 6. The fractal dimension is calculated for a window of length w. In the current embodiment, the value of w is 5, the number of sample points along a scanline, although that value can be varied. The fractal dimension is calculated from the difference between the maximum RF signal value in the window centered at a given depth, r, and the minimum of that same window. The length of the window, w, is added to this difference, and the result is then normalized with the length of the window. The logarithm of that result is then divided by the logarithm of the ratio of the total number of samples in a scanline, n, to the length of the window. The calculation of the fractal dimension at each depth along a scanline is shown in Equation 10. This fractal dimension measure is calculated for the central n-w samples in a scanline.

After the measurements of the fractal dimension have been calculated based on the ultrasound signal, the thickness of the bladder wall may be calculated. The following calculations correspond to the seventh block 58 of FIG. 6.

The fractal dimension, fd, of the RF signal in the region of the bladder muscle wall is then modeled as a parabolic equation as a function of depth, r. The model of the equation for a single depth point is given in Equation 11. In that equation, there are 3 parameters (a, b, and c) that define the parabola with the depth along a scanline r, and the addition of a random element ε. The subscript i indicates a specific value of r, ƒd, and ε.

Equation 11.

ƒdᵢ=arᵢ²+brᵢ+c+εᵢ

An equation of the form in Equation 11 is obtained for each depth point in the region of the wall. The number of observations is variable and depends on the thickness of the bladder wall as observed by the ultrasound signal. Assuming a set of n observations, the subscript i would count the observations from 1 to n. The set of n equations of the form in Equation 11 may be compressed into a matrix equation given in Equation 12. Each row of the fd, and ε, and the X matrix correspond to one of the n observations. The parabola parameters of Equation 11 are collected in the vector β.

$$fd = X β + ε \text{where} \\ fd = [ \begin{matrix}{fd}_{1} \\ {fd}_{2} \\ ⋮ \\ {fd}_{n}\end{matrix} ] , X = [ \begin{matrix}{r}_{1}^{2} & {r}_{1} & 1 \\ {r}_{2}^{2} & {r}_{2} & 1 \\ ⋮ & ⋮ & ⋮ \\ {r}_{n}^{2} & {r}_{n} & 1\end{matrix} ] , β = [ \begin{matrix}a \\ b \\ c\end{matrix} ] , \text{and} \\ ε = [ \begin{matrix}{ϵ}_{1} \\ {ϵ}_{2} \\ ⋮ \\ {ϵ}_{n}\end{matrix} ] . \qquad \text{Equation 12}$$

The next step is to estimate the values of the parameters of the parabola in the set of n equations of the form in Equation 11 or in the matrix Equation 12 based on the set of observations. A least-squares estimation of the parameters is used, and the calculation for these estimates is shown in Equation 13. In Equation 13, the t superscript indicates matrix transpose, and the −1 superscript indicates the matrix inverse. Parameters with hats (\hat{ }) indicate that the value is the least-squares estimate of those parameters.

Equation 13.

\hat{β}=(X^{t}X)⁻¹X^{t}fd

The estimates of the parabola parameters (\hat{β}=└â \hat{b} ĉ┘^{t}) can be substituted into the parabola model to calculate the estimated fractal dimension at each depth r, as shown in Equation 14. The location of the maximum fractal dimension can be determined by setting the first derivative of the parabola model to equal 0 (Equation 15) and solving for r. The location where the fractal dimension is maximal is given in Equation 16.

$$\begin{matrix}f \hat{d} ( r ) = \hat{a} {r}^{2} + \hat{b} r + \hat{c} . & \text{Equation 14} \\ \frac{ⅆ f \hat{d} ( r )}{ⅆ r} = 2 \hat{a} r + \hat{b} = 0. & \text{Equation 15} \\ {r}_{{fd}_{max}} = - \frac{\hat{b}}{2 \hat{a}} . & \text{Equation 16}\end{matrix}$$

To determine the maximal fractal dimension as defined by the parabolic model, simply substitute Equation 16 into Equation 14 and solve for fdₘₐₓ. The resulting value is shown in Equation 17.

$$f {\hat{d}}_{max} = \frac{- {\hat{b}}^{2} + 4 \hat{c}}{4 \hat{a}} . \qquad Equation 17$$

To determine the locations where the fractal dimension is 97% of the maximum value, multiply Equation 17 by 0.97, substitute the result into Equation 14 and solve for r using the quadratic formula. The locations where the fractal dimension is 97% of the maximum value, r_{97%}, are given in Equation 18.

$${r}_{97 \%} = \frac{- \hat{b} \pm \sqrt{{\hat{b}}^{2} - 4 \hat{a} ( \hat{c} + 0.97 \frac{{\hat{b}}^{2} + 4 \hat{c}}{4 \hat{a}} )}}{2 \hat{a}} . \qquad Equation 18$$

Two values for r_{97%} will be calculated from Equation 18. The difference between those two values will identify the thickness of the bladder muscle wall along the given scanline. Since these scanlines may or may not be perpendicular to the bladder muscle surface and bladder wall thickness must be measured along a line perpendicular to the bladder surface, a collection of these measurements are combined to determine the actual thickness of the bladder wall.

These measurements could be made at any surface of the bladder muscle wall. In FIG. 8, three scanlines (a first scanline 36, a second scanline 40, and a third scanline 44) are shown to cross the bladder muscle in two locations: the anterior wall closest to the transducer, and the posterior wall furthest from the transducer. The dotted portion of the lines represents the portion of the scanplanes that passes through the bladder muscle wall. The first 36, the second 40, and third 44 scanlines are shown transmitting through the subserosal wall location 72 and submucosal wall location 74. The parabolic model described previously can be applied twice on each to determine the thickness of both the anterior and posterior wall. The maximum and minimum and mean values of these thicknesses are used in the mass calculation and historical tracking of data. In the embodiment shown, this final thickness determination marks the end of the process identified in the seventh block 58 of FIG. 6.

In the preferred embodiment, the bladder is assumed to have a uniform wall thickness, so that a mean wall thickness value is derived from the scanned data and used for the bladder mass determination. Only three scanlines are shown in a plane, each separated by 1.5 degrees from each other. Both the number of scanlines in the plane and the angles separating each scanline within a plane may be varied.

Bladder mass determination. Once the thickness and the surface area have been measured, the mass of the bladder may be calculated. The volume of muscle tissue is assumed to be the surface area times the wall thickness, where the assumption is based on a uniform wall thickness at all points around the bladder. The mass is then the product of the volume of muscle tissue, the specific gravity of the bladder muscle tissue and the density of water. The specific gravity of bladder muscle is a known value readily available in medical reference texts. In the embodiment shown, this mass calculation corresponds to the eighth block 59 of FIG. 6.

In an alternate embodiment, the methods to obtain the wall-thickness data and the mass data via downloaded digital signals can be configured by the microprocessor system for remote operation via the Internet web-based system. The Internet web-based system (“System For Remote Evaluation Of Ultrasound Information Obtained By A Program Application-Specific Data Collection Device”) is described in co-pending and commonly assigned patent application Ser. No. 09/620,766, herein incorporated by reference. The internet web-based system has multiple programs that collect, analyze, and store organ thickness and organ mass determinations. These alternate embodiments thus provides an ability to measure the rate at which internal organs undergo hypertrophy with time and permits disease tracking, disease progression, and the provision of educational instructions to patients and caregivers.

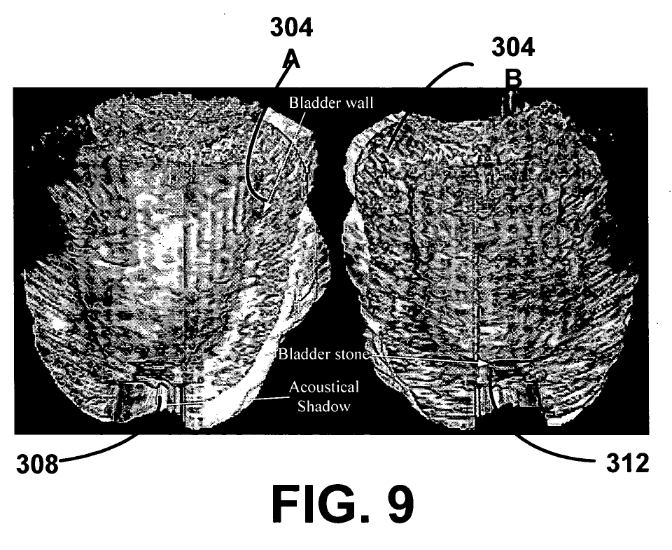

FIG. 9 depicts a substantially bas-relief 2D presentation volume rendering of the left (first) and right (second) half bladder hemisphere views of a bladder. The first and second hemispheric views are virtual images that provides to the physician the similar look of a bladder as seen with an optical cystoscope and provides a non-invasive means to diagnose bladder-related programs using the volume renderings from image processing of digitized ultrasound echoes presented in the image cones. The image cones are either the 3D arrays of 2D scanplanes, or the 3D scan cone of 3D distributed scanlines. The image processing includes algorithms to normalize unbalanced intensity distributions along a scanline (General Software Time Gain Control), normalize ultrasound echo variation caused by differences in surface reflectivity (Reverberation Control), normalize ultrasound conduction differences between fluid regions and surrounding tissues (Underneath Fluid Compensation), and a 3D viewing software tool to present the substantially bas-relief 2D presentation. The image processing algorithms to obtain the bas-relief 2D presentation volume rendering is described in co-pending and commonly assigned provisional Patent Application Serial No. 60/470,525 (“Ultrasound Virtual Cystoscope System and Method”), herein incorporated by reference. The left bladder hemisphere shows a bladder wall 304A and an artifact of ultrasound imaging, an acoustical shadow 308. Similarly, the right bladder hemisphere shows a bladder wall 304b, and a simulated bladder stone 312 artificially added to the data set.

The acoustical shadow 308 ultrasound artifact is put to good use by the system and method of the invention to foster the visualization of the simulated bladder stone 308. Near the acoustical shadow 308 is a set of low-resolution vertical lines that delineates the simulated bladder stone 312. The white arrowhead in FIG. 9 points to a single vertical line near the region underneath the simulated bladder stone 312 along the scanline where the region around the acoustical shadow 308 is imaged near the simulated bladder stone 312.

While the preferred embodiment of the invention has been illustrated and described, as noted above, many changes can be made without departing from the spirit and scope of the invention. Accordingly, the scope of the invention is not limited by the disclosure of the preferred embodiment.

The embodiments of the invention in which an exclusive property or privilege is claimed are defined as follows:

What is claimed is:

1. A method to determine bladder wall thickness using an ultrasound transceiver, the method comprising:
positioning an ultrasound transceiver exterior to a patient such that at least a portion of the bladder wall is within the range of the transceiver;
transmitting radio frequency ultrasound pulses to, and receiving those pulses echoed back from, the external and internal surface of the portion of the bladder wall; and, based on those pulses
calculating for the portion of the bladder wall
(a) the surface area of the external and internal surfaces, and
(b) the distance between the external and internal surfaces.

2. The method of claim 1, wherein the radio frequency ultrasound pulses are sent to the bladder in one or more of the forms selected from the group consisting of a scanplane, a spiral, and a random line.

3. The method of claim 2, wherein the form selected is a scanplane, and the scanplane is associated with an array, the array selected from the group consisting of a translational array, a wedge array, and a rotational array.

4. The method of claim 3, wherein the scanplane in the array is selected from the group consisting of uniformly spaced, non-uniformly spaced, and a combination of uniformly spaced and non-uniformly spaced scanplanes.

5. The system of claim 4, wherein the scanplane comprises a plurality of scanlines, the scanlines selected from the group consisting of uniformly space, non-uniformly spaced, and a combination of uniformly space and non-uniformly spaced scanlines.

6. The system of claim 5, wherein the uniform spacing between each scanplane is approximately 7.5 degrees.

7. The system of claim 5, wherein the uniform spacing between each scanline is approximatel 1.5 degrees.

8. The method of claim 1, wherein the echoes are classified into latitudinal and longitudinal components.

9. The method of claim 8, wherein the latitudinal and longitudinal components of the echoes reflecting back from the area of the portion of the bladder wall is defined to be S, and comprises a plurality of surface patches, sᵢⱼ, where i and j represent the latitude and longitude components, such that the area of S of the portion of the bladder wall is the sum of the plurality of patches, S=Σsᵢⱼ.

10. The method of claim 9, wherein the surface patch sᵢⱼ is further defined by a vector sᵢⱼ(u,v)=xᵢⱼ(u,v)i+yᵢⱼ(u,v)j+zᵢⱼ(u,v)k, where i, j, k, are unit vectors in the x-, y-, and z-directions respectively, and u and v are surface patch coordinates.

11. The method of claim 1, wherein the thickness separating the surface areas is ƒdr determined from the relationship
$${fd}_{r} = \frac{log ( \frac{max ( {RF}_{r = r - w / 2 , r + w / 2} ) - min ( {RF}_{r = r - w / 2 , r + w / 2} ) + w}{w} )}{log ( \frac{n}{w} )}$$
wherein the terms max (RF_{r=r-w/2, r+w/2}) and min (RF_{r=r-w/2, r+w/2})+w refer to the maximum and minimum radio frequency (RF) value for a window of length w, centered at a given depth, r, along a scanline of a given number of samples, n, such that the fractal dimension is calculated from the difference between the maximum radio frequency (RF) signal value in the window centered at a given depth, r, then normalized with a total number of samples in a scanline, n.

12. The method of claim 11, wherein the thickness separating the inner and outer wall area ƒdr is adjusted by a parabolic function of the form is determined from the relationship ƒdᵢ=arᵢ²+brᵢ+c+εᵢ, where there are 3 parameters (a, b, and c) that define a parabola function with the depth along a scanline r, and the addition of a random element ε, wherein the subscript i indicates a specific value of r, ƒd, and ε.

13. The method of claim 12, wherein the parabolic function is at least 97% of the maximal value of a fractal dimension is determined from the relationship
$${r}_{97 \%} = \frac{- \hat{b} \pm \sqrt{{\hat{b}}^{2} - 4 \hat{a} ( \hat{c} + 0.97 \frac{{\hat{b}}^{2} + 4 \hat{c}}{4 \hat{a}} )}}{2 \hat{a}} ,$$
where the parameters with hats (\hat{ }) indicate that the value is the least-squares estimate of those parameters.

14. The method of claim 1, wherein the area each bladder wall is determined for bladders containing approximately 0 ml to approximately 1000 ml.

15. A method to determine bladder wall mass using an ultrasound transceiver, the method comprising:
positioning an ultrasound transceiver exterior to a patient such that at least a portion of the bladder wall is within the range of the transceiver;
transmitting radio frequency ultrasound pulses to, and receiving those pulses echoed back from, external and internal surface of the portion of the bladder wall; and, based on those pulses
calculating for the portion of the bladder wall
(a) the surface area of the external and internal surfaces of the bladder wall,
(b) the thickness between the surfaces areas, and
(c) the mass between the surface areas.

16. The method of claim 15, wherein the radio frequency ultrasound pulses are sent to the organ in a plurality of forms, the forms selected from the group consisting of a scanplane, a spiral, and a random line.

17. The method of claim 16, wherein the scanplane is associated with an array, the array selected from the group consisting of a translational array, a wedge array, and a rotational array.

18. The method of claim 17, wherein the scanplane of the rotational array includes a plurality of scanlines, the scanplane further being separated from adjacent scanplanes by approximately 7.5 degrees, and the scanline within the scanplane being separated from adjacent scanlines by approximately 1.5 degrees.

19. The method of claim 15, wherein the mass of the portion of the bladder wall is calculated as a function of the calculated surface area and thickness.

20. A system for determining the mass of an internal organ, the system comprising:
a transceiver configured to deliver radio frequency ultrasound pulses in a plurality of scanplanes to the organ of a patient, receive echoes of the pulses reflected from the organ, and process the echoes into horizontal and vertical components; and
a computer system in communication with the transceiver, the computer system having a microprocessor and a memory, the memory further containing stored programming instructions operable by the microprocessor to determine the surface area, thickness, and mass of the organ based on the horizontal and vertical components of the echoes.

21. The system of claim 20, wherein the radio frequency ultrasound pulses are sent to the organ in a plurality of forms, the forms selected from the group consisting of a scanplane, a spiral, and a random line.

22. The system of claim 21, wherein the scanplane is associated into an array, the array selected from the group consisting of a translational array, a wedge array, and a rotational array.

23. The system of claim 22, wherein the scanplane of the rotational array includes a plurality of scanlines, the scanplane further being separated from adjacent scanplanes by approximately 7.5 degrees, and the scanline within the scanplane being separated from adjacent scanlines by approximately 1.5 degrees.

24. The system of claim 20, wherein the organ is a bladder.

25. The system of claim 20, wherein the transceiver includes a display to present the graphic image of a scanplane in two-dimensions and the plurality of scanplanes in three-dimensions.

26. The system of claim 20, wherein the stored programming instructions in the memory further comprise a plurality of equations to calculate bladder wall surface area and bladder wall thickness at one or more of anterior, posterior, or lateral locations of the bladder wall.

27. The system of claim 26, wherein the plurality of equations to measure bladder wall surface area include
$$\begin{matrix}{s}_{i , j} ( u , v ) = [ \begin{matrix}\begin{matrix}{x}_{i , j} ( u , v ) \\ {y}_{i , j} ( u , v )\end{matrix} \\ {z}_{i , j} ( u , v )\end{matrix} ] , \\ A ( S ) = {\sum }_{i , j} A ( {s}_{i , j} ) , \\ A ( {s}_{i , j} ) = {\sum }_{i , j} \frac{1}{4} ( \begin{matrix}{\vert \frac{\partial {s}_{i , j}}{\partial u} \times \frac{\partial {s}_{i , j}}{\partial v} \vert}_{u = \frac{3 - \sqrt{3}}{6} , v = \frac{3 - \sqrt{3}}{6}} + \\ {\vert \frac{\partial {s}_{i , j}}{\partial u} \times \frac{\partial {s}_{i , j}}{\partial v} \vert}_{u = \frac{3 - \sqrt{3}}{6} , v = \frac{3 + \sqrt{3}}{6}} \\ {\vert \frac{\partial {s}_{i , j}}{\partial u} \times \frac{\partial {s}_{i , j}}{\partial v} \vert}_{u = \frac{3 + \sqrt{3}}{6} , v = \frac{3 - \sqrt{3}}{6}} + \\ {\vert \frac{\partial {s}_{i , j}}{\partial u} \times \frac{\partial {s}_{i , j}}{\partial v} \vert}_{u = \frac{3 + \sqrt{3}}{6} , v = \frac{3 + \sqrt{3}}{6}}\end{matrix} ) , and \\ \vert \frac{\partial {s}_{i , j}}{\partial u} \times \frac{\partial {s}_{i , j}}{\partial u} \vert = \sqrt{\begin{matrix}{( \frac{\partial {y}_{i , j}}{\partial u} \frac{\partial {z}_{i , j}}{\partial v} - \frac{\partial {z}_{i , j}}{\partial u} \frac{\partial {y}_{i , j}}{\partial v} )}^{2} + \\ {( \frac{\partial {z}_{i , j}}{\partial u} \frac{\partial {x}_{i , j}}{\partial v} - \frac{\partial {z}_{i , j}}{\partial u} \frac{\partial {x}_{i , j}}{\partial v} )}^{2} + \\ {( \frac{\partial {x}_{i , j}}{\partial u} \frac{\partial {y}_{i , j}}{\partial v} - \frac{\partial {y}_{i , j}}{\partial u} \frac{\partial {x}_{i , j}}{\partial v} )}^{2}\end{matrix}} .\end{matrix}$$

28. The system of claim 27, wherein the equations used to calculate bladder wall thickness include
$$\begin{matrix}{fd}_{r} = \frac{log ( \frac{max ( {RF}_{r = r - w / 2 , r + w / 2} ) - min ( {RF}_{r = r - w / 2 , r + w / 2} ) + w}{w} )}{log ( \frac{n}{w} )} , and \\ {fd}_{i} = {ar}_{i}^{2} + {br}_{i} + c + {ϵ}_{i} , and \\ {r}_{97 \%} = \frac{- \hat{b} \pm \sqrt{{\hat{b}}^{2} - 4 \hat{a} ( \hat{c} + 0.97 \frac{{\hat{b}}^{2} + 4 \hat{c}}{4 \hat{a}} )}}{2 \hat{a}} .\end{matrix}$$

29. The system of claim 20, wherein the bladder wall mass is calculated as a product of bladder wall area, thickness, and specific gravity of the bladder wall.

30. The system of claim 29, wherein the specific gravity of the bladder wall is approximately 0.96.

31. The system of claim 30, wherein bladder thickness is a mean of a plurality of bladder wall thicknesses calculated based on a plurality of scanned bladder locations.

32. The system of claim 20, wherein the computer system is configured for remote operation via an Internet web-based system, the internet web-based system having a plurality of programs that collect, analyze, and store organ thickness and organ mass determinations, such that the rate at which internal organs undergo hypertrophy may be determined.

33. The system of claim 20, wherein the plurality of programs further include instructions to permit disease tracking, disease progression, and provides educational instructions to patients.

34. A system for determining the mass of an internal organ, the system comprising:
a transceiver configured to deliver radio frequency ultrasound pulses in a plurality of scanplanes to the organ of a patient and receive echoes of the pulses reflected from the organ; and
a means for determining the surface area of the organ and the thickness of the organ based on the echoes received by the transceiver, and further to calculate the mass of the organ as a function of the organ thickness and area.

35. The system of claim 34, wherein the transceiver is adjusted for the anatomy of male and female patients.

36. The system of claim 34, wherein the means for determining the surface area of the organ include a first plurality of equations to analyze the echoes received by the transceiver, the first plurality of equations including:
$$\begin{matrix}{s}_{i , j} ( u , v ) = [ \begin{matrix}\begin{matrix}{x}_{i , j} ( u , v ) \\ {y}_{i , j} ( u , v )\end{matrix} \\ {z}_{i , j} ( u , v )\end{matrix} ] ; \\ A ( S ) = {\sum }_{i , j} A ( {s}_{i , j} ) ; \\ A ( {s}_{i , j} ) = {\sum }_{i , j} \frac{1}{4} ( \begin{matrix}{\vert \frac{\partial {s}_{i , j}}{\partial u} \times \frac{\partial {s}_{i , j}}{\partial v} \vert}_{u = \frac{3 - \sqrt{3}}{6} , v = \frac{3 - \sqrt{3}}{6}} + \\ {\vert \frac{\partial {s}_{i , j}}{\partial u} \times \frac{\partial {s}_{i , j}}{\partial v} \vert}_{u = \frac{3 - \sqrt{3}}{6} , v = \frac{3 + \sqrt{3}}{6}} \\ {\vert \frac{\partial {s}_{i , j}}{\partial u} \times \frac{\partial {s}_{i , j}}{\partial v} \vert}_{u = \frac{3 + \sqrt{3}}{6} , v = \frac{3 - \sqrt{3}}{6}} + \\ {\vert \frac{\partial {s}_{i , j}}{\partial u} \times \frac{\partial {s}_{i , j}}{\partial v} \vert}_{u = \frac{3 + \sqrt{3}}{6} , v = \frac{3 + \sqrt{3}}{6}}\end{matrix} ) ; and \\ \vert \frac{\partial {s}_{i , j}}{\partial u} \times \frac{\partial {s}_{i , j}}{\partial u} \vert = \sqrt{\begin{matrix}{( \frac{\partial {y}_{i , j}}{\partial u} \frac{\partial {z}_{i , j}}{\partial v} - \frac{\partial {z}_{i , j}}{\partial u} \frac{\partial {y}_{i , j}}{\partial v} )}^{2} + \\ {( \frac{\partial {z}_{i , j}}{\partial u} \frac{\partial {x}_{i , j}}{\partial v} - \frac{\partial {z}_{i , j}}{\partial u} \frac{\partial {x}_{i , j}}{\partial v} )}^{2} + \\ {( \frac{\partial {x}_{i , j}}{\partial u} \frac{\partial {y}_{i , j}}{\partial v} - \frac{\partial {y}_{i , j}}{\partial u} \frac{\partial {x}_{i , j}}{\partial v} )}^{2}\end{matrix}} .\end{matrix}$$

37. The system of claim 34, wherein the means to determine the thickness of the organ include a second plurality of equations to analyze the echoes received by the transceiver, the second plurality of equations including
$$\begin{matrix}{fd}_{r} = \frac{log ( \frac{max ( {RF}_{r = r - w / 2 , r + w / 2} ) - min ( {RF}_{r = r - w / 2 , r + w / 2} ) + w}{w} )}{log ( \frac{n}{w} )} , \\ {fd}_{i} = {ar}_{i}^{2} + {br}_{i} + c + {ϵ}_{i} , and \\ {r}_{97 \%} = \frac{- \hat{b} \pm \sqrt{{\hat{b}}^{2} - 4 \hat{a} ( \hat{c} + 0.97 \frac{{\hat{b}}^{2} + 4 \hat{c}}{4 \hat{a}} )}}{2 \hat{a}} .\end{matrix}$$

38. The system of claim 34, wherein the mass of the organ is determined as a product of the surface area, the thickness, and the density of the organ.

39. A system for displaying an internal organ and determining the wall thickness of the internal organ, the system comprising:
a transceiver configured to deliver radio frequency ultrasound pulses to the organ of a patient and receive echoes of the pulses reflected from the organ; and,
a computer system in communication with the transceiver, the computer system having a microprocessor and a memory, the memory further containing stored programming instructions operable by the microprocessor to
a) present a 3-D rendering of the organ, and
b) determine the thickness of the organ based on the echoes received by the transceiver.

40. The system of claim 39, wherein the 3-D rendering comprises:
an image segmentation process;
an image enhancement process to correct ultrasound echo artifacts;
a 2-D region of interest selection process;
a 3-D interpolation process; and
a 3-D projection process on a 2-D image.

41. The system of claim 40, wherein the segmentation process comprises thresholding one or more scanlines.

42. The system of claim 40, wherein the image enhancement process to correct ultrasound echo artifacts comprises:
a general software time gain control algorithm to normalize ultrasound echo strength variations due to differences in the depth of reflecting surfaces;
a reverberation control algorithm to distinguish ultrasound echoes caused by differences in ultrasound surface reflectivity; and
an underneath fluid compensation algorithm to normalize ultrasound echo strength variations due to differences in ultrasound conduction between fluid regions and surrounding tissues.

43. The system of claim 40, wherein the 2-D region of interest selection process comprises delineating pixels within a scanplane to define a region of interest.

44. The system of claim 40, wherein the 3-D interpolation process comprises selecting the pixel within the region of interest of a scanplane to define 3-D voxels spanning the region between pixels of regions of interest between adjacent scanplanes.

45. The system of claim 40, wherein the 3-D projection process upon a 2-D image comprises distributing the 3-D voxels upon the 2-D image in bas-relief using a software rendering tool.

46. The system of claim 39, wherein the organ is a bladder.

47. A method to determine bladder wall thickness using an ultrasound transceiver, the method comprising:
positioning an ultrasound transceiver exterior to a patient such that at least a portion of the bladder wall is within the range of the transceiver;
transmitting radio frequency ultrasound pulses as a plurality of three-dimensional distributed scanlines to, and receiving those pulses echoed back from, the external and internal surface of the portion of the bladder wall; and, based on those pulses
calculating for the portion of the bladder wall
a) the surface area of the external and internal surfaces, and
b) the distance between the external and internal surfaces.

48. The method of claim 47, wherein the plurality of three-dimensional distributed scanlines form a scan cone having a plurality of image signals of the bladder.

49. The method of claim 48, wherein the image signals are processed by a computer system in communication with the transceiver, the computer system having a microprocessor and a memory, the memory further containing stored programming instructions operable by the microprocessor to present a 3-D rendering of the bladder based upon the image signals.

50. The system of claim 49, wherein the 3-D rendering comprises:
an image segmentation process;
an image enhancement process to correct ultrasound echo artifacts;
a 2-D region of interest selection process;
a 3-D interpolation process; and
a 3-D projection process on a 2-D image.

51. The system of claim 50, wherein the segmentation process comprises thresholding one or more scanlines.

52. The method of claim 51, wherein the 3-D projection process of the bladder in the scan cone forms two approximately equal hemispheres as a 2-D bas-relief image.

53. The method of claim 52, wherein the bladder hemispheres present a cross-sectional depiction of the bladder wall, the internal structures within the bladder, and the internal structures within and the thickness of the bladder wall.
